(19) United States
(12) Patent Application Publication
(10) Pub. No.: US 2004/0054371 A1
(43) Pub. Date: Mar. 18, 2004

(54) Bone screw

(76) Inventors: Michael Dierks, Wermelskirchen (DE); Peter Kretschmer, Bruchkobel (DE)

Correspondence Address: Steven J. Grossman, Grossman, Tucker, Perreault & Pfleger, PLLC, 55 South Commercial Street, Manchester, NH, 03101, US

(21) Appl. No.: 10/461,250

(22) Filed: Jun. 11, 2003

(30) Foreign Application Priority Data
Jun. 11, 2002 (DE) 202 09 025.6

(51) Int. Cl. A61B 17/58

(52) U.S. Cl. 606/73

(57) ABSTRACT

The invention relates to a bone screw (1) with a screw shank (2) that can be fastened in a bone, particularly a vertebra, and a screw head (3), on which a fastening device (20) can be fastened that can be. swiveled as regards its inclination relative to the screw shank (2) and/or rotated and that displays a receptacle (26) for mounting a connecting rod. To permit swiveling of the fastening device relative to the screw shank over the largest possible angular range, as well as easy and reliable handling, the screw head (3) displays a recess (5), to which a swiveling element (6) that can be located in the recess and at least swiveled and fixed in position relative to the screw shank can be assigned, to which a fastening device (20) can be fastened. The recess (5) can be located on the screw head (3) at the level of the screw shank (2) and designed as a slot that can extend parallel to the longitudinal axis of the screw shank (2).

The invention relates to a bone screw with a screw shank that can be fastened in a bone, particularly a vertebra, and a screw head, on which a fastening device with a receptacle for mounting a connecting rod can be fastened, where the fastening device can be swiveled as regards its inclination relative to the screw shank and/or rotated. The invention further relates to a bone screw and an adapter for a device according to the invention.

Various bone screws of this kind, or devices encompassing them, are known. For example, the use of pedicle screws with ball-like screw head is known, on which a one or two-part clamp is mounted, by means of which a connecting rod can be clamped in place and fixed in different swiveling positions relative to the screw shank. However, the swiveling range of the connecting rod relative to the screw shank is relatively limited in this context. Furthermore, generic bone screws are known, in which swiveling of the clamping device relative to the screw shank is possible over a larger angular range, but in which, during use of the bone screw or the device assigned to it, the clamping device can unintentionally move a relatively long distance away from its target position, or unintentional dismantling of the clamping device can occur as a result of unintentional detachment of the means for fixing the position of the clamping device relative to the screw shank.

The object of the invention is to provide a generic bone screw, which permits swiveling and/or rotation of a fastening device relative to the screw shank over the largest possible angular range and which can be handled easily and reliably. Furthermore, the object of the invention is to provide an adapter for a bone screw of this kind.

According to the invention, the object is solved by a bone screw in which the screw head is provided with a recess, in which a swiveling element can be located that can be swiveled and fixed in position relative to the screw shank and to which a fastening device can be fastened. Because the swiveling element is mounted in a recess in the screw head, the swiveling angle can be limited by the dimensions of the width of the recess in the swiveling direction, meaning that the swiveling element cannot move out of its target position to an undesirable degree. Moreover, this permits a particularly compact and stable design of the bone screw, and of the overall arrangement with associated adapter and fastening device, in the region of the adapter and the fastening area for the connecting rod. Given suitable alignment of the connecting rod, the swiveling axis can run parallel to the longitudinal axis of the connecting rod, in which context the axis of rotation and the longitudinal axis of the connecting rod can at all times enclose an angle relative to each other during rotating motion, preferably an angle of 90 degrees. Furthermore, in unlocked state, in which swiveling over the entire swiveling angle is possible, the swiveling element can be fastened reliably and in captive fashion on the screw. For this purpose, the swiveling element can, for example, be provided with a broader area which can, for example, be located on its head end, or the locking means described below, or other suitable means, can be provided.

The object is moreover solved by providing an adapter in accordance with the further, independent claim. The invention is developed by the characteristics of the sub-claims. The invention encompasses both a bone screw and a bone screw with elements assigned to it, and particularly located on it in accordance with the intended use, such as a swiveling element, an adapter and/or a fastening device, which together at least partially or completely form a device for mounting a connecting rod of a spinal fixing element. For the sake of simplicity, the invention will be described below on the basis of the device, in which context it must be understood that the respective developments each also relate to an isolated bone screw.

The fastening device for fixing the connecting rod, which can be located on the device according to the invention or is encompassed by it, can, in particular, be designed as a clamping device, without being limited to this. To this end, a clamping means, such as a locking screw, can be provided, by means of which the connecting rod can be fastened to the device according to the invention. The fastening means can act indirectly on the connecting rod or on an extension thereof, for example by way of a collar band. The fastening means, particularly in the form of a clamping means, can also act directly on a fastening area of the connecting rod, which can, for example, be designed in the form of an eye through which the swiveling element can be passed, so that the fastening area of the connecting rod can be pressed against the device according to the invention. In this context, the eye can be of open or closed design at the side and possibly provided with teeth on one or both sides. The fastening device can also act in some other suitable way, preferably in a positive and/or non-positive manner.

The swiveling element can be designed to swivel primarily or exclusively in a monoaxial manner in one direction in space about a defined axis in the recess, and can display a lateral guide, preferably a bilateral guide, so that the swiveling element can swivel with only little or virtually no lateral play and/or with only little or essentially no lateral tilting motion. However, the swiveling element may possibly also be capable of swiveling in two or more directions enclosing an angle relative to each other, for instance in the area of a spherical calotte. The lateral limitation of the swiveling motion and/or sliding motion of the swiveling element, which can each take place without play or with little play, can be achieved by the lateral edges of the recess or by other suitable means.

The swiveling angle can be ≧±5°, preferably ≧±25°, for instance ≧±45°, without being limited to this. The neutral position of the swiveling axis of the swiveling element, i.e. the position of the swiveling element at maximum swivel towards the longitudinal axis of the screw shank, can be within an angle of 0° to 45° relative to the longitudinal axis of the screw shank. At an angle of 0°, the swiveling element thus forms the axial extension of the screw shank.

The swiveling axis of the swiveling element preferably includes an angle of 90° to the longitudinal axis of the screw shank.

The recess in the screw head for receiving the swiveling element can be of pocket-like design, especially in the form of a blind hole, so that the edges of the recess limit the swiveling angle and/or unilateral or bilateral motion of the swiveling element. The swiveling element can be inserted into the blind hole from the front for fixing on the screw head. If necessary, the swiveling element can act on retaining means of the screw head, to which end the swiveling element can be rotatable in such a way that it reaches behind one or more projections located on the screw, particularly in the area of the recess. If necessary, the recess for receiving the swiveling element can also be designed as a through-hole with an opening at the rear on the screw head, or as a laterally open receptacle, so that the swiveling element can be inserted into the screw head in a direction other than the direction in which the swiveling element projects outwards from the screw head, especially the opposite direction. In this context, the swiveling element can, for example, be of essentially pin-shaped or also U-shaped design. The swiveling element can also engage two adjacent recesses in the screw head, which either face each other or open in different, e.g. opposite, directions. The recesses can, for example, be provided on opposite sides of the screw head.

The receptacle for the swiveling element is preferably located at the level of the screw shank.

A lateral, preferably bilateral, guide for the swiveling element is preferably provided which, in unlocked state of the swiveling element, acts on it so as to protect it, without play or with little play, from lateral sliding and/or swiveling.

Furthermore, a separate retaining element is preferably provided, which secures the unlocked and/or locked swiveling element against removal from the receptacle, especially by longitudinal displacement, or retains it there. This separate retaining element is preferably provided in addition to the means for fixing the position of the swiveling element relative to the screw shank, and can be operated independently of it, such that the swiveling element is securely retained on the screw head, even in the event of complete removal of the element that fixes the position of the swiveling element in its selected swiveling position. The fixing element can be the fastening means for fixing the connecting rod, such as the tightening nut described further below, without being limited to this. This substantially improves the handling of the device according to the invention, since unintentional detachment of the swiveling element from the screw is prevented even when the swiveling element is completely unlocked. The securing means can pass through or reach into the swiveling element, or it can also simply reach behind the swiveling element, thus protecting it from falling out in the direction of insertion into the screw head, for example.

The separate retaining element can be designed as a retaining pin or retaining bolt, which is inserted into an opening in the screw head that runs into the recess for receiving the swiveling element, so that the separate retaining element can act on the swiveling element. The recess for receiving the separate retaining element is preferably designed as a through-hole ending on both sides of the screw head, preferably on opposite face sides. The face sides can be the face ends of a screw head designed as a transverse bar. In this context, both ends of the separate retaining element lie flush with the face sides of the screw head, preferably completely closing off the insertion hole. At the same time, the separate retaining element can define the swiveling axis of the swiveling element.

The separate retaining element, which can in particular be designed as a retaining pin, can be fixed on the screw head in a manner preventing sliding and/or rotation, to which end suitable non-positive and/or positive means or suitable other means can be provided, for instance in the form of a screw thread. Another suitable retaining means can also be provided instead of a retaining pin.

The separate retaining element can secure the swiveling element against longitudinal displacement, preferably in any of the intended swiveling positions, where longitudinal displacement is prevented completely or partially by the separate retaining element. To this end, the swiveling element can display a hook-like projection, preferably an eye, which at least partly reaches behind the separate retaining element, or into which the separate retaining element can be inserted. In this context, the inside diameter of the eye preferably corresponds to the diameter of the engaging retaining pin. Preferably, the diameter of the separate retaining element in the area in which it contacts the swiveling element corresponds to the diameter of the separate retaining element in one or both of its end areas, for instance in the form of a pin having a constant diameter over its entire length.

The end of the swiveling element projecting outwards from the screw head can display a retaining area for fixing a fastening device and/or an adapter positionable between the fastening device and the screw head, where the fastening device can in either case be designed as a clamping device. The retaining area can be provided with a threaded section for this purpose.

The screw head is preferably designed as an extension that extends to one or both sides from the screw shank and can extend perpendicularly or at a different angle relative to the shank, or as a spherical head, without being limited to this. The screw head preferably forms the part of the pedicle screw that is the greatest axial distance away from the screw shank. Regardless of the other features of its design, the screw head can be fastened to the shank in one piece or in detachable manner.

Particularly preferably, at least the partial area of the screw head on which the adapter and/or the fastening device can be fixed, or the entire screw head, is designed in the form of a body of revolution, or of a preferably outwardly curved partial section of the same. The body of revolution preferably displays a principal axis with a greater length than axes in other directions, where the swiveling element can swivel about this principal axis. The body of revolution can, for example, be designed as a section of a cylinder, such that the peripheral surface of the screw head forms a cylinder surface or partial cylinder surface, where the swiveling axis of the swiveling element is parallel or coaxial to the cylinder axis. For this purpose, it is sufficient if areas of the screw head on one or both sides and/or on the upper and/or lower side of the receptacle for the swiveling element are provided in the form of cylinder surfaces, such that an adapter and/or a clamping device located on the swiveling element can perform swiveling motion about the screw head axis while maintaining contact with one or more cylinder surfaces. In this context, the recess for receiving the swiveling element is preferably made in a cylinder surface or partial cylinder surface of the screw head. Preferably, at least the area of the screw head that is covered by the adapter and/or the swiveling device at the maximum swiveling angle, is designed as a cylinder surface. In terms of design, it is particularly simple if the screw head as a whole is designed as a cylindrical rod. In this context, the face sides of the screw head can be designed to be plane and parallel to each other, or in another suitable manner. The same applies if the extension has the shape of a different body of revolution, such as a barrel-shaped body, an ellipsoid of revolution or the like.

Where appropriate, the screw head can also be designed as part of a spherical surface in order to permit swiveling of the swiveling element in one or more directions in space, where the recess for receiving the swiveling element is preferably made in the two-dimensional section of the spherical surface.

In a preferred embodiment, an adapter is provided, which can be fixed on the swiveling element and displays a contact surface that can be positioned in linear fashion, or preferably fully, against the screw head and that is preferably designed as a convexly curved surface, as well as another contact surface for a fastening device, particularly a clamping deice, so that the fastening device can be fastened on the adapter if needed. The concave, indented surface can be a partial surface of a body of revolution that preferably has a principal axis with a greater length than axes in other directions, e.g. a partial surface of a cylinder or a partial surface of a barrel, ellipsoid of revolution or the like. The indentation of the adapter facing the screw head can be designed in partially cylindrical form. Independently thereof, or in combination therewith, the adapter surface opposite the contact surface of the adapter on the screw head can be of plane design. The description relating to the shape of the adapter surface can apply similarly to the contact surface of a fastening element, e.g. a clamping element such as a U-shaped clip, on the screw head.

The contact surface of the adapter assigned to the fastening device, on which the fastening device can be positioned with a contact surface, can essentially be of plane, spherically convex, spherically concave or conical (male or female cone) design. The principal axis of the convex, concave or conical sphere is preferably arranged parallel to the longitudinal axis of the swiveling element. The curvature in space of the corresponding contact surface of the fastening element can be the same as, or different to, the contact surface of the adapter.

The fastening device, particularly a clamping device, is preferably designed to rotate about an axis relative to the adapter, where the axis of rotation is preferably arranged coaxially to the longitudinal axis of the swiveling element. If appropriate, the axis of rotation of the fastening device and the longitudinal axis of the swiveling element can also enclose an angle relative to each other, or be offset relative to each other.

Preferably, the adapter displays a through-hole for passage of the swiveling element, and the fastening device a receptacle for the fastening area of the swiveling element, which can also be designed as a through-hole. As a result, the adapter and the fastening device can be slid onto the swiveling element and swiveled together relative to the screw shank. In this context, the adapter permits large-area contact both on the curved screw head surface and on the fastening device.

If appropriate, the fastening device can also display an arc-shaped surface, particularly in the form of a section of the periphery of a body of revolution, such as a cylinder or an ellipsoid of revolution, which can be positioned against the screw head surface such that the fastening device can also be fixed directly on the screw head.

Preferably, at least one, or both, of the respective facing contact surfaces of screw head and adapter and/or adapter and fastening device are roughened, particularly toothed, thus increasing the resistance of the specified components to rotation relative to each other. In particular, the two contact surfaces of the adapter on the screw head and the fastening element can be roughened, particularly toothed. This is particularly expedient in cases where the fastening device is fixed directly on the screw head and only relatively small contact surface engage each other, for instance when the contact surfaces display different curvatures in space. One or both of the contact surfaces of the above-mentioned pairs of surfaces can, however, also be of essentially smooth design.

In particular, the contact surface of the adapter for the screw head and/or the fastening element can be roughened, particularly toothed or radially toothed, or also of smooth design. Independently hereof, or simultaneously hereto, the contact surfaces of the fastening device and/or the screw head assigned to the adapter can be roughened, particularly toothed, or also of smooth design.

In particular, the contact surface of the adapter facing the fastening device can display radial toothing, particularly if the adapter contact surface is of plane, spherically concave or spherically convex shape. The contact surface can also be of rough design, particularly if it is of plane, spherically concave or spherically convex shape. The contact surface can also be of smooth design, particularly if it is of plane, spherically concave or spherically convex shape. The contact surface can also display toothing, particularly radial toothing, or be of smooth or rough design if it is of male conical or female conical shape. The corresponding contact surface of the fastening device can in each case display toothing, particularly radial toothing, or be of smooth or rough design, particularly given a respectively corresponding shape of the adapter contact surface, this permitting linear or full contact with each other in each case.

Preferably, the extension of the adapter and/or the fastening device in the swiveling direction is such that the receptacle for the swiveling element is completely covered by the adapter and/or the fastening device in every possible swiveling position.

The adapter and/or the fastening device can extend over the entire width of the screw head, without being limited to this.

Particularly preferably, the fastening device is designed as a clamping device, where tensioning of the clamping device for fixing the connecting rod is achieved by the same clamping means as used to fix the position of the adapter and/or clamping device about the swiveling axis. To this end, a clamping element, such as a straining screw, can, for example, be provided, which can be fixed on a threaded section of the swiveling element in such a way that operation of the straining screw tensions the clamping device, simultaneously applying force to it relative to the adapter, whereby force is applied to the adapter relative to the screw head and the swiveling element thus fixed in position on the screw head. If appropriate, separate locking means can also be provided for this purpose.

In the device according to the invention, the screw head can be positioned eccentrically to the screw shank, meaning that the swiveling axis is laterally offset relative to the longitudinal axis of the screw shank. The swiveling axis can be located inside the diameter of the screw shank or outside it. The lateral offset can be up to ¼, up to ½ or up to the full radius or diameter of the screw shank at the level of the screw head, without being limited to this. The lateral offset can be less or more than 1 mm, in the range from approx. 1 mm to approx. 10 mm, or up to approx. 20 mm, preferably in the range from approx. 3 mm to approx. 5 mm, without being limited to this. The eccentricity is preferably chosen such that, when the fastening device contacts the vertebra and is positioned between the spinous and transverse process, the space remaining between the outer side of the fastening device and the spinous process is such that the fastening device can be freely fitted at every level of the spinal column without restriction in any case and thus without implantation-induced traumatization of the spinous processes.

An example of the invention is described below and explained on the basis of the figures. The drawings show the following:

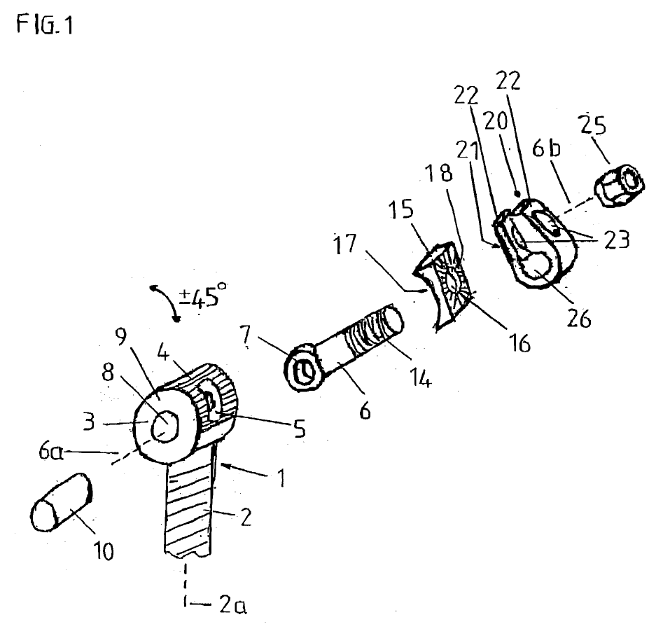

FIG. 1 An exploded view of a first practical example of a bone screw according to the invention with assigned components,

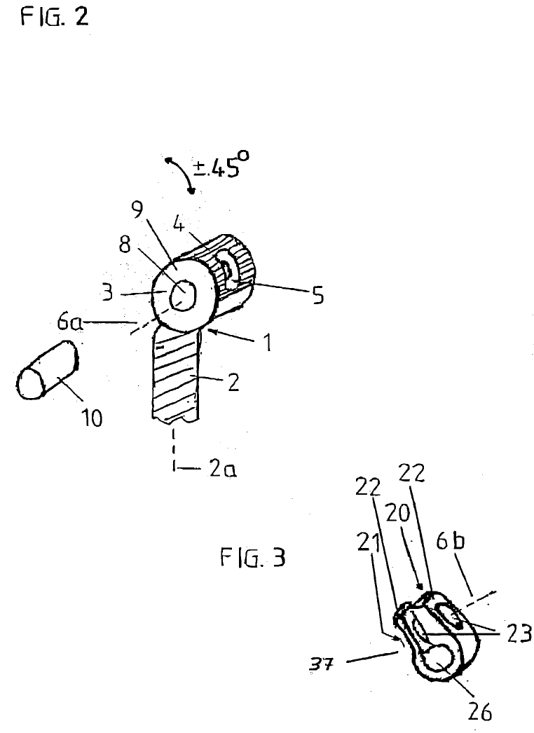

FIG. 2 A further practical example of a bone screw according to the invention,

FIG. 3 A further practical example of a clamping device for a bone screw according to the invention.

FIG. 1 shows a bone screw 1 according to the invention as part of a device for mounting a connecting rod of a spinal fixing means, where the bone screw displays a shank 2 and a screw head 3. The screw head is designed in the manner of a cylindrical crossbar, which is located perpendicular to longitudinal axis 2a of the shank and projects from shank 2 on both sides. A recess 5 is provided in cylindrical peripheral surface 4 of the screw head in the form of a slot, into which the end of rod-like swiveling element 6 can be inserted. The swiveling element can be swiveled through an angle of approx. ±45 degrees in the recess, enclosing an angle of approx. 90 degrees with shank 2 when in its maximum swiveling position facing shank 2. The neutral position of the axis of the swiveling element, in which the swiveling element is swiveled to the maximum relative to the longitudinal axis of the screw shank, is at an angle of approx. 20° relative to the longitudinal axis of the screw shank, i.e. sloping upwards, as illustrated in FIG. 1. In this context, the width of recess 5 is dimensioned such that swiveling element 6 makes virtually play-free contact with both lateral borders of recess 5, or is a slight distance away from them.

To captively mount swiveling element 6 in a manner preventing sliding, it is provided with an eye 7 on the end locatable in recess 5. Furthermore, screw head 3 is provided with a through-hole 8 extending in the longitudinal direction, which ends on both face sides 9 of head 3 and intersects recess 5. The hole in eye 7 can be positioned flush with through-hole 8 in this context, meaning that a locking bolt 10 can be inserted into through-hole 8 and passed through eye 7, where the locking bolt ends flush with both face sides 9, or can project beyond or stand back from one or both face sides. Locking bolt 10 can be fixed on screw head 3 non-positively, by means of a thread or by other suitable means in a manner preventing sliding. The outside diameter of bolt 10 in the connecting area corresponds to the inside diameter of eye 7, meaning that the swiveling element is fixed on the screw head in the longitudinal direction, preferably essentially without play, or with little play. Bolt 10 can be inserted, from one or both face sides 9 of screw head 3, into the corresponding hole, which can be designed as a blind hole or as a through-hole, where bolt 10 completely closes off the insertion hole.

Moreover, the device according to the invention displays an adapter 15, which displays a through-hole 16, through which swiveling element 6 passes, and, on surface 17 opposite screw head 3, a cylindrical indentation, such that the full surface of the adapter can be positioned against the cylindrical peripheral surface 4 of screw head 3 and guided in a swiveling motion in contact with this surface. The opposite surface 18 of the adapter is of plane design and can be positioned against the plane underside 21 of the fastening element for a connecting rod, said element being designed as clamp 20 according to the practical example. The clamp is designed as a U-shaped clip in this instance, but it can also be designed in some other, suitable way in order to fix the connecting rod. The clamp can be rotated about direction 6b in this context. The two legs 22 of the clamp are provided with through-holes 23 and can be fitted onto swiveling element 6, the end of which is provided with a threaded section 14. Tightening nut 25 can be screwed onto threaded section 14, thereby squeezing the two legs 22 together and clamping a connecting rod located in receptacle 26 in place. At the same time, nut 25 applies force on the clamp against the adapter and on the adapter against cylindrical peripheral surface 4 of the screw head, as a result of which the swiveling element, and thus also the adapter and the clamping device, are simultaneously fixed in position on the screw head. To increase the angular stability, either one or both of the corresponding contact surfaces 4, 17 and 18, 21 of the screw head and the adapter and of the adapter and the clamp are provided with toothing, the corresponding contact surface being smooth or rough. It goes without saying that other embodiments of the surface finish may also be expedient.

A connecting rod can easily be positioned optimally on the screw by swiveling the swiveling element in unlocked state and rotating the clamp about the longitudinal axis of the swiveling element.

The embodiment illustrated in FIG. 2 essentially corresponds to that shown in FIG. 1, identical components being identified by the same reference numbers.

In contrast to FIG. 1, screw head 3, which is of rod-shaped design according to the practical example, has a principal axis that is offset relative to longitudinal axis 2a of the screw shank. In this context, the principal axis corresponds to the longitudinal axis of the screw head and the swiveling axis 6a of the adapter or the fastening device. In this instance, the lateral offset corresponds approximately to the radius of the screw shank.

The fastening device according to FIG. 3 represents a clamping device in the form of a U-shaped clip, the underside of which that faces the screw head has an indentation not in the form of a spherical calotte if the U-shaped clip is adapted to the contour of the screw head and can be brought into contact, particularly full-area contact, with the screw head. The surface defining indentation 37 corresponds to a section of an outer surface of a non-spherical body of revolution, such as a cylinder or an ellipsoid of revolution. The indentation is preferably designed in such a way that its full surface can be positioned against a body of revolution, such as a cylinder or an ellipsoid of revolution, where the body of revolution is not spherical and preferably displays a principal axis that is longer than the other axes. Due to the cylindrical-concave indentation according to the practical example, the fastening device can make linear or full-surface contact with the screw head or an adapter and be laterally supported on it, this being impossible in the case of contact between a spherical head and an indentation in the form of a spherical calotte. The indentation can, for example, be provided on a one or two-leg fastening device for a connecting rod.

Instead of the cylindrical peripheral surfaces of the screw head and the adapter and/or the clamping device, it is also possible in each case for one or all of the corresponding contact surfaces to be designed as sections of a spherical surface, meaning that the swiveling element can perform swiveling motion in several directions enclosing an angle relative to each other, or that the adapter and/or the clamping device can be swiveled relative to the screw head in several directions enclosing an angle.

The fact that the swiveling element in the device according to the invention or in the bone screw according to the invention can, during the swiveling motion, perform motion along the recess in the screw head, which is preferably open towards the swiveling plane, and undergo an arc-shaped change in position in the recess, preferably in the form of an arc of a circle, results in special advantages of the subject matter of the invention.

Bone Screw

List of Reference Numbers

1 Bone screw

2 Shank

2a Longitudinal axis

3 Head

4 Peripheral surface

5 Recess

6 Swiveling element

6a Swiveling axis

6b Direction

7 Eye

8 Through-hole

9 Face side

10 Locking bolt

14 Threaded section

15 Adapter

16 Through-hole

17, 18 Surface

20 Clamp

21 Underside

22 Leg

23 Through-hole

25 Nut

26 Receptacle

37 Indentation

1. Bone screw (1) with a screw shank (2) that can be fastened in a bone, and a screw head (3) on which a fastening device (20) with a receptacle (26) for mounting a connecting rod can be fixed, where the fastening device can be swiveled as regards its inclination relative to the screw shank (2) and/or rotated, characterized in that the screw head (3) displays a recess (5), in which a swiveling element (6) that can at least be swiveled and fixed in position relative to the screw shank can be located for fastening an adapter (15) and/or a fastening device (20) for fixing a connecting rod and/or a fastening area of a connecting rod.

2. Bone screw according to claim 1, characterized in that the recess (5) is designed as a slot.

3. Bone screw according to claim 2, characterized in that the slot extends parallel to the longitudinal axis of the screw shank (2).

4. Bone screw according to one of claims 1 to 3, characterized in that the recess (5) is located on the screw head (3) at the level of the screw shank (2)

5. Bone screw according to one of claims 1 to 4, characterized in that a lateral guide for an assignable swiveling element (6) is provided, which interacts with the swiveling element (6) in unlocked state of said swiveling element (6).

6. Bone screw according to one of claims 1 to 5, characterized in that a separate retaining element (10) is provided, which secures an assignable swiveling element (6) against removal from the recess (5) when in unlocked state.

7. Bone screw according to claim 6, characterized in that the retaining element (10) can be inserted into a hole (8) ending in the recess (5), at least up to the recess (5), and in that the retaining element (10) can be fixed on the end section of an assignable swiveling element (6) located in the recess (5).

8. Bone screw according to claim 6 or 7, characterized in that the separate retaining element (10) is designed in such a way that a projection of a swiveling element (6) locatable in the recess (5) can be reached behind by the retaining element, or engage the swiveling element (6).

9. Bone screw according to one of claims 1 to 8, characterized in that a swiveling element (6) is assigned to the screw that displays a fastening area for fastening an adapter (15) or a fastening device (20) for fixing a connecting rod.

10. Bone screw according to one of claims 1 to 9, characterized in that the screw head (3) displays the form of a non-spherical body of revolution, at least in the contact area for positioning an adapter and/or a fastening device (20) for fixing a connecting rod.

11. Bone screw according to one of claims 1 to 10, characterized in that the screw head (3) is located eccentrically relative to the screw shank (2).

12. Bone screw according to one of claims 1 to 11, characterized in that an adapter is assigned to the screw, which can be located between the screw head of the bone screw and a fastening device (20) for fixing a connecting rod and/or a fastening area of a connecting rod.

13. Bone screw according to claim 12, characterized in that the adapter (15) displays a contact surface (18) assigned to a fastening device (20), which is of plane, spherically convex, spherically concave or conical design.

14. Bone screw according to one of claims 1 to 13, characterized in that a fastening device (20) for fixing a connecting rod is also encompassed.

15. Bone screw according to one of claims 1 to 14, characterized in that a swiveling element (6) with a fastening area (14) is provided, to which an adapter (15) or a fastening device (20) for fixing a connecting rod can be fixed, and in that a fastening means (25) is provided, by means of which the fastening device (20) can be operated and, at the same time, the adapter (15) or the fastening device (20) can be fixed against the screw head (3), thereby fixing the position of the swiveling element.
